(19) United States
(12) Patent Application Publication
(10) Pub. No.: US 2015/0230848 A1
(43) Pub. Date: Aug. 20, 2015

(54) SELF-EXPANDING BONE STABILIZATION DEVICES

(71) Applicant: SPINEALIGN MEDICAL, INC., San Jose, CA (US)

(72) Inventors: Paul E. CHIRICO, Campbell, CA (US); Benny M. CHAN, San Jose, CA (US); Alison M. SOUZA, Santa Clara, CA (US); Peter G. KNOPP, Pleasanton, CA (US)

(21) Appl. No.: 14/704,737

(22) Filed: May 5, 2015

Related U.S. Application Data
(63) Continuation of application No. 12/650,851, filed on Dec. 31, 2009.
(60) Provisional application No. 61/142,122, filed on Dec. 31, 2008.

(51) Int. Cl. A61B 17/88

(52) U.S. Cl. CPC A61B 17/8858; A61B 2017/00867

(57) ABSTRACT

Described herein are bone implant having bifurcated struts for distracting and supporting bone, as well as systems and methods for treating bone using them. These self-expanding implants for repairing bone may include two or more bifurcated struts that extend from a central body or shaft in the deployed configuration. The stabilization device may be attached to an inserter. An inserter may be used to hold the stabilization device in a collapsed delivery configuration so that it can be inserted into bone.

### CROSS-REFERENCE TO RELATED APPLICATIONS

This application is a continuation application of U.S. patent application Ser. No. 12/650,851 filed Dec. 31, 2009, which claims priority to Provisional Application Ser. No. 61/142,122, filed on Dec. 31, 2008, titled “SELF-EXPANDING BONE STABILIZATION DEVICES”.

This application may also be related to U.S. application Ser. No. 12/041,607, filed Mar. 3, 2008, titled “FRACTURE FIXATION SYSTEM AND METHOD”; U.S. application Ser. No. 12/044,884, filed Mar. 7, 2008, titled “TRANSDISCAL INTERBODY FUSION DEVICE AND METHOD”; U.S. application Ser. No. 12/044,880, filed Mar. 7, 2008 titled “SYSTEMS, METHODS AND DEVICES FOR SOFT TISSUE ATTACHMENT TO BONE”; U.S. application Ser. No. 12/024,938, filed Feb. 1, 2008, titled “SYSTEMS, DEVICES AND METHODS FOR STABILIZING BONE”; U.S. application Ser. No. 12/025,537, filed Feb. 4, 2008, titled “METHODS AND DEVICES FOR STABILIZING BONE COMPATIBLE FOR USE WITH BONE SCREWS”; and U.S. application Ser. No. 11/468,759, filed Aug. 30, 2006, titled “IMPLANTABLE DEVICES AND METHODS FOR TREATING MICRO-ARCHITECTURE DETERIORATION OF BONE TISSUE”.

All of these applications are herein incorporated by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference in its entirety.

### FIELD OF THE INVENTION

Described herein are systems, devices, and methods for treating and supporting bone. The invention also relates to systems, devices, and methods for treating and supporting cancellous bone within vertebral bodies, particularly vertebral bodies which have suffered a vertebral compression fracture (VCF).

### BACKGROUND OF THE INVENTION

Deterioration of bone tissue, and particularly micro-architecture deterioration, can result from a variety of factors including disease, aging, stress and use. For example, osteoporosis is a disease characterized by low bone mass and micro-architecture deterioration of bone tissue. Osteoporosis leads to bone fragility and an increase fracture risk. The World Health Organization defines osteoporosis as a bone density more than 2.5 standard deviations below the young adult mean value. Values between 1 and 2.5 standard deviation below the young adult mean are referred to as osteopenia.

In an osteoporotic bone, pores or voids in the sponge-like cancellous bone increase in dimension, making the bone very fragile. Although bone breakdown occurs continually as the result of osteoclast activity in young, healthy bone tissue, this breakdown is balanced by new bone formation by osteoblasts. In contrast, in an elderly patient, bone resorption can surpass bone formation, resulting in deterioration of bone density. Osteoporosis occurs largely without symptoms until a fracture occurs.

Repair of osteoporotic bone fractures, as well as other bone fractures, may require expansion and support of the bone and affiliated structures. Thus, it may be beneficial to include an implant that is capable of being easily inserted into the bone without causing or requiring further damage to the bone and other tissues, and that is also capable of being expanded (or allowed to self-expand) into a supporting configuration that provides long-lasting support to the bone. For example, related U.S. application Ser. No. 12/024,938 (filed on Feb. 1, 2008), titled “SYSTEMS, DEVICES AND METHODS FOR STABILIZING BONE”) published as US 2008/0281364 A1 on Nov. 13, 2008, describes bone stabilization devices and methods for inserting them using a delivery device. The described delivery device may be configured to include a cannula (or multiple cannula) and one or more trocars. In performing such procedures it would be extremely beneficial to have a stabilization device that has an enhanced strength and stability in the self-expanded configuration. In particular, devices that expand from a narrow (e.g., tubular) collapsed form into an expanded position having a principle and secondary expanded form.

Described herein are examples of devices, systems including devices, and methods of using them that are configured to provide enhanced support and long-term stability. In particular, the devices described herein are configured to be inserted in a strain-distributing manner that reduces the risk of implant failure with enhanced ease of use and operation.

### SUMMARY OF THE INVENTION

Described herein are improved bone stabilization devices. Stabilization devices are typically self-expanding devices, and the control may regulate the self-expansion so that the rate and degree of self-expansion allowed is regulated. The devices may be lockable, and may include a latch or other locking structure. The devices described herein typically expand to form a device having arcuate struts or arms extending between the proximal and distal end regions; the angular spacing between adjacent arcuate struts or arms is typically not equal. For example, the angle between adjacent struts may alternate between a minor angle and a major angle. When four arcuate struts or arms are present, for example, the angles between adjacent struts or arms may be 30 degrees and 150 degrees, or 40 and 140 degrees, etc., in which the angle that is less than 90 degrees is the minor angle, and the angle that is greater than 90 degrees is the major angle. Generally, when n (e.g., 3, 4, 5, etc.) arcuate struts or arms are present on the device, the minor angle are defined by angles <(360/n), and the major angles are defined by >(360/n).

Illustrated below are examples in which there are four arcuate struts (arms) that extend from a collapsed state when the device is not deployed, into an expanded or deployed state. The material from which the devices are made may be any self-expanding material, including shape memory materials such as Nitinol and the like. The devices may be configured to use with a delivery system or device, including those described in the related applications. For example, the devices may include one or more attachment region (including threaded regions) at the proximal and distal end.

Any of these devices may be pre-biased in the expanded state, so that they can be delivered in the collapsed state under tension. For example, the delivery device may provide tension to keep them collapsed, by applying tension to separate the proximal and distal ends of the device. This may allow them to be deployed by releasing the tension on them. In some variation, the devices are deployed so that the distal end remains in a relatively stable position within the anatomy while the proximal end is moved towards the distal end. This may prevent the device from extending further into the anatomy (e.g., bone cavity) than is desired during deployment. The delivery device may be adapted so that the device is deployed (expanded) by allowing the proximal end of the device to move distally while keeping the distal end relatively stable.

In general, the devices described herein may be formed by forming (e.g., cutting, molding, etc.) four or more slots into a tube of material. Alternatively, the slots may be formed by cutting a sheet of material that is rolled into a tube. In some variations, the struts are formed by attaching individual struts to a proximal and distal end.

When the struts are formed by cutting slots or channels, the slots may be formed of different lengths and/or widths, or at different regions along the perimeter of the tube. If the devices are formed by cutting the material, it may be modified thereafter to form the pre-biased expanded shape. The device may also be modified to include the attachment (e.g., threaded) regions at either or both the proximal and distal ends. For example, a distal insert including a threaded region may be secured into the distal end of the tube. The distal insert may be a cylinder having a threaded inner region that matches the threading on the applicator device. The outer surface of the distal insert mates with the inner surface of the distal end of the device. Thus, the distal insert may be welded (e.g., soldered, tack welded, swaged, etc.) into the device. In some variations, the distal end of the device is threaded with threads complementary to the outer surface of the distal insert. These treads may be oriented opposite to the threads within the distal insert. The distal insert may be formed of any appropriate material, including steel, titanium, aluminum, Nitinol, etc.

During processing to form the device, it may be treated pre-bias in the expanded configuration. The device may also be processed to prevent corrosion, or to otherwise enhance biocompatibility. For example, in one variation the device is initially cut from a tube of shape memory material by laser cutting. The slots forming the arcuate struts may be cut in any desired arrangement and number. Thereafter, the device may be deburred/deslugged. Heat shaping/treatment shaping may then be performed to bias the device in the expanded shape. Thereafter the surface may be treated by removing any oxide, electropolishing, coating, etc. A distal insert may be added (e.g., by welding) to the distal end. The device may again be electropolished, and the attachment sites (e.g., to the applicator) formed, for example, by threading the device. Finally, the device may be passivated to prevent corrosion.

For example, described herein are bone stabilization implants that are pre-biased to self-expand from a collapsed delivery configuration to an expanded deployed configuration within cancellous bone. A bone stabilization implant may include: an elongate body having a proximal end and a distal end and an inner passage extending through the elongate body from the proximal end to a distal end region; a plurality of bifurcated support struts configured to extend from the elongate body, wherein each bifurcated strut is configured to extend from the elongate body in the deployed configuration and to separate into two or more sub-struts extending longitudinally from a portion of the strut; a proximal attachment region having a first releasable attachment configured to attach to an inserter; and a distal attachment region having a second releasable attachment configured to attach to the inserter.

Any appropriate attachment region may be used for the proximal and/or distal attachment regions. For example, the attachment region may be a threaded region (that mates with another attachment region on the inserter), the attachment region may be a cut-out region (or notched region), including an L-shaped cut out, an S-shaped cut out, a J-shaped cut out, or the like, into which a pin, bar, or other structure on the inserter may mate. In some variations, the attachment region is a hook or latch. The attachment region may be a hole or pit, with which a pin, knob, or other structure on the inserter mates. In some variations, the attachment region includes a magnetic or electromagnetic attachment (or a magnetically permeable material), which may mate with a complementary magnetic or electromagnet region on the inserter. In each of these variations the attachment region on the device mates with an attachment region on the inserter so that the device may be removably attached to the inserter.

The attachment regions may be located at the distal or proximal ends (or near the distal or proximal ends), on the outside of the body, on the inside of the body, or passing through the body. For example, in one variation, the proximal attachment region of the bone stabilization device is located within the inner passage of the elongate body. In one variation, the proximal attachment region is a threaded region within the inner passage of the elongate body. In one variation, the distal attachment region is within the inner passage of the elongate body. The distal attachment region may be a threaded region within the inner passage of the elongate body.

In some variations the proximal and distal attachment regions are configured so that they may be independently operated to attach/release the implant to the inserter. For example, the attachment regions may be formed of threaded regions within the inner passage of the elongate body that are threaded in opposite directions. The attachment regions may be oppositely-oriented attachment regions of the same type (e.g., threaded regions, notched regions, etc.) or of different types. In some variations the attachment regions are separately lockable.

The stabilization implant may include two or more bifurcated support struts. In general the support struts are bifurcated longitudinally (e.g., along the length of the implant) so that only a portion of the support strut is bifurcated. In some variations only one or some of the support struts are bifurcated. The support struts may be formed by cuts made in the body of the implant. For example, the support struts may be formed by substantially longitudinal slits through the implant. The support struts may be formed along the majority of the length of the implant, or only partially along the length of the implant. The support struts (and the sub-struts extending therefrom) may be positioned symmetrically on the implant (e.g., so that they extend to form a maximum diameter of the expanded implant near the center of the length of the implant) or they may be positioned asymmetrically along the length of the implant.

For example, a bone stabilization implant may include sub-struts that are configured to extend from the elongate body asymmetrically relative to the length of the elongate body. In some variations, the sub-struts are arranged to extend more from the distal end of the length of the elongate body. In some variations, the sub-struts extend longitudinally for less than 90% of the length of the support strut (or less than 95%, less than 85%, less than 80%, less than 75%, less than 70%, less than 50%, etc.), to form the bifurcated support strut.

Also described herein are bone stabilization implants that are pre-biased to self-expand from a collapsed delivery configuration to an expanded deployed configuration within cancellous bone comprising: an elongate body having a proximal end and a distal end and an inner passage extending through the elongate body from the proximal end to a distal end region; a plurality of bifurcated support struts configured to extend from the elongate body, wherein each bifurcated strut is configured to extend from the elongate body in the deployed configuration and separate into two or more sub-struts extending longitudinally from less than 90% of the length of the strut; a proximal attachment region in the inner passage of the elongate body having a first releasable attachment configured to attach to an inserter; and a distal region having a second releasable attachment configured to attach to the inserter.

Also described herein is a bone stabilization implant pre-biased to expand from a collapsed delivery configuration to an expanded deployed configuration within cancellous bone, the bone stabilization implant comprising: an elongate body having a proximal end and a distal end and an inner passage extending through the elongate body from the proximal end to a distal end region; a plurality of bifurcated support struts configured to extend from the elongate body and separate along part of their length into two or more sub-struts, wherein the angles between adjacent sub-struts on each bifurcated support strut is less than the angle between adjacent sub-struts on different bifurcated support struts; a proximal attachment region having a first releasable attachment configured to attach to an inserter; and a distal attachment region having a second releasable attachment configured to attach to the inserter.

Also described herein are stabilization systems comprising: an implant inserter having a first elongate member and a second elongate member (wherein the first and second elongate member are slideably coupled) and a stabilization implant pre-biased to expand from a collapsed delivery configuration to an expanded deployed configuration within cancellous bone. Any of the bone stabilization implants described herein may be used, including a bone stabilization implant comprising: an elongate body having a proximal end and a distal end and an inner passage extending through the elongate body from the proximal end to a distal end region;

a plurality of bifurcated support struts configured to extend from the elongate body and separate along part of their length into two or more sub-struts; a proximal attachment region configured to releasable attach to the first elongate member of the inserter; and a distal attachment region configured to releasably attach to the second elongate member of the inserter.

The inserter may also include a lock configured to axially lock the first elongate member relative to the second elongate member. The first elongate member of the inserter may be slideably disposed within the second elongate member.

Also described herein is a method of treating a bone with a pre-biased stabilization implant having an elongate body with a proximal and distal end and a plurality of bifurcated support struts configured to extend from the elongate body and separate into two or more sub-struts, the method comprising: delivering the pre-biased stabilization implant within a cancellous bone in a collapsed configuration; and allowing the implant to self-expand within the cancellous bone so that the sub-struts cuts through the cancellous bone and secure the implant within the bone. The step of allowing the device to self-expand may comprise allowing the proximal end of the implant to move towards the distal end of the implant while the distal end of the implant remains relatively fixed with respect to the bone.

### BRIEF DESCRIPTION OF THE DRAWINGS

Although the figures below contemplate specific embodiments, other variations are included, and may be understood from these drawings and the accompanying description.

FIG. 1 is a side perspective view of one variation of an asymmetric (radially asymmetric) self-expanding device as described herein.

FIG. 2 is a side view of the device of FIG. 1, showing the different size slots cut in the tube to form the arcuate struts/arms. This variation includes two bifurcating struts which each have two ‘sub-struts’, as shown in FIG. 4 (showing the expanded configuration of the device).

FIG. 3 is a perspective end view of the device of FIG. 1.

FIG. 4 is a perspective view of the device of FIG. 1 in the expanded configuration, showing the four (two sets of two) struts.

FIG. 5 is a side perspective view of another variation of a self-expanding implant, as described herein. In this variation the struts are formed by channels of two different lengths; a longer length and a shorter length. The cuts are arranged so that the struts will be positioned more closely to the end of the device facing the right in FIG. 5.

FIG. 6 is a side view of the device of FIG. 5, above.

FIG. 7 is another side perspective view of the device of FIG. 5, showing the opposite end, including a distal insert having inner threads. These threads may be used to attach to the inserter/applicator and/or to other devices, including screws (e.g., pedicle screws). The proximal end may also include a similar threaded region, or other attachment structure(s) (not visible in FIG. 7).

FIG. 8 shows a pseudo-colored (shown as grayscale in the figure) image illustrating the stress seen on the expanded variation of the device shown in FIG. 5. The device is shown as a side perspective view of the device (implant) shown in FIG. 5.

FIGS. 9A and 9B illustrate one variation of an expandable implant having bifurcated struts, showing the major and minor angles between the adjacent struts in the expanded form. In this example, the implants are shown end-on; FIG. 9A shows the device form the proximal end, and FIG. 9B shows the device from the distal end.

FIG. 10 illustrates one variation of an implant inserter.

FIG. 11 shows the implant insert of FIG. 10 with an implant attached.

FIGS. 12A-12D illustrate one method of using an implant having bifurcated struts, as described herein.

### DETAILED DESCRIPTION

The devices, systems and methods described herein may aid in the treatment of fractures and microarchitetcture deterioration of bone tissue, particularly vertebral compression fractures (“VCFs”). The implantable stabilization devices described herein (which may be referred to as “stabilization implants” or simply “implants”) may help restore, e.g., restore height, and/or augment bone. Thus, the stabilization devices described herein may be used to treat pathologies or injuries. For purposes of illustration, many of the devices, systems and methods described herein are shown with reference to the spine. However, these devices, systems and methods may be used in any appropriate body region, particularly bony regions. For example, the methods, devices and systems described herein may be used to treat hip bones.

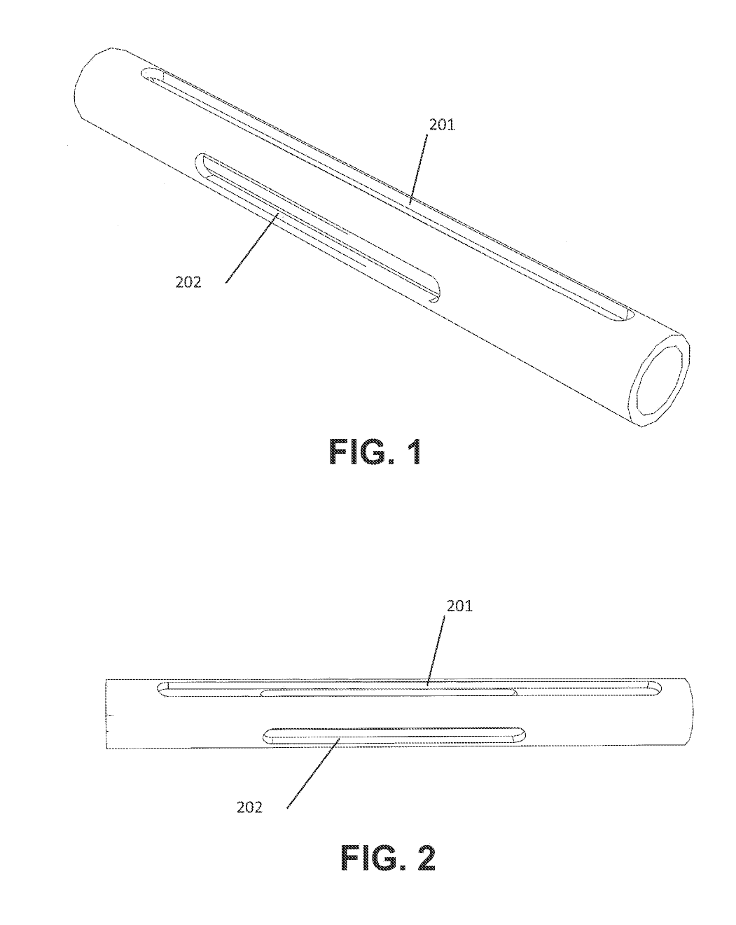

The stabilization implants described herein may be self-expanding, and may be pre-biased to self-expand from a compressed profile having a relatively narrow diameter (e.g., a delivery configuration) into an expanded profile (e.g., a deployed configuration). The stabilization implant generally includes a body or shaft region having a plurality of bifurcated struts that may extend from the shaft body. The distal and proximal regions of a stabilization implant may include one or more attachment regions configured to attach to an inserter for inserting (and/or removing) the stabilization device from the body. FIGS. 1 through 9B illustrate exemplary stabilization devices.

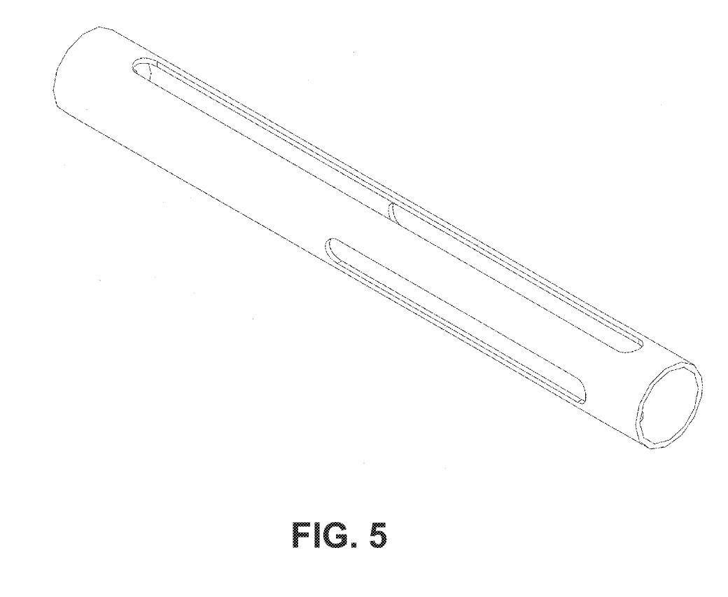

For example, FIG. 1 shows one variation of an implant having bifurcated support struts. This implant has an elongate (tubular) body that is sized to completely fit within the cancellous bone of a human vertebra (e.g., it may be less than a few inches in length, less than 1.75 inches, less than 1.5 inches, less than 1.25 inches, less than 1 inch, etc.). This implant is hollow, and contains cuts in the body that will form the arcing support struts and sub-struts. In the collapsed (e.g., insertion) configuration the implant may require force to maintain it in the collapsed configuration, since the device may be pre-biased in the delivery (expanded) configuration as shown in FIG. 4. Thus, in the collapsed configuration the implant may need to be connected to an insert (not shown) and held or locked in the collapsed configuration.

The variation shown in FIG. 1 has a tubular body, although it does not need to be tubular. The elongate body may have non-circular cross-sections as well, including oval cross-sections, square cross-sections, triangular (or other flat-sided) cross-section, or the like. In addition, although the struts are formed by making slots in the body, they need not be. In some variations, the slots are not open (as shown in FIGS. 1-3), but are closed. In other variations, the struts are formed separately and attached together to form the elongate body, or are attached to other portions of the elongate body.

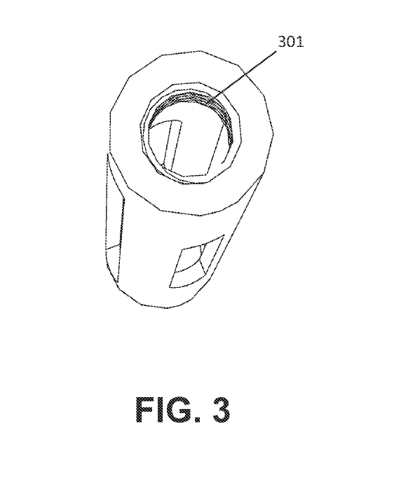

FIG. 2 shows the variation shown in FIG. 1 in side view. The cut 201 into the body between the two major (or support) struts is shown on the top. Shorter longitudinal cuts in the support struts will form the sub-struts. For example, the cut 202 shown at the bottom of FIG. 2.

The embodiment shown in FIGS. 1-4 is also hollow, and open at both the distal and proximal ends. In some variations, the proximal end is open while the distal end is closed. In other variations, the distal end includes an anchor or bone-grasping end that is configured to secure it in place once positioned in the bone along its length (not shown). For example, the distal end region may include spikes, pins, or other extendable/expandable grip regions other than the struts. This may facilitate expansion of the implant from the proximal end while leaving the distal end substantially stationary relative to the bone.

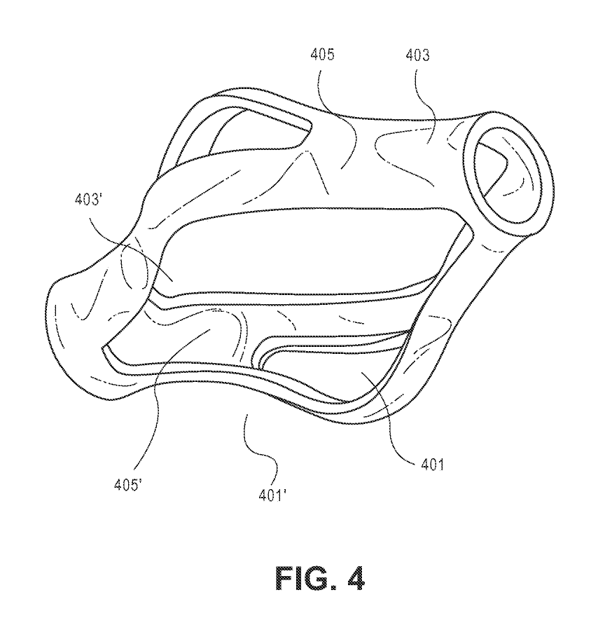

In general, the distal and proximal end regions of the implant may include their own attachment sites to an inserter, as illustrated below. FIG. 3 shows an end view of the implant in perspective, in which the inner surface of the passageway through the device includes releasable attachment region 301 that is configured to attach to an inserter (not shown). This variation of a releasable attachment region is a threaded region into which a complementary region on the inserter may screw into and attach. In this variation, threaded region is formed from an insert that is secured into the inner diameter of the implant (within the inner passage), but in some variations the attachment region is integrally formed in (e.g. cut from) the body of the implant. In some variations the attachment site maybe located on the outside of the body of the implant, or pass through the body of the implant. Any appropriate attachment site (e.g., notch, pin, etc.) may be used.

The proximal attachment site may also be adapted to connect to other devices or implants, in addition to an inserter. For example, the proximal attachment site may be adapted to mat with a screw, such as a pedicle screw. Thus, the implant may act as an anchor for a pedicle screw.

Similarly the distal end region may have an attachment site configured to releasably and/or lock-ably connect to the inserter. The connector at the distal end region may also be any appropriate connector, including mechanical connectors (threads, pins, notches, etc.), electromagnetic connectors, or the like. In general, the connector at the proximal and distal end allow the inserter to independently control the proximal and distal ends of the device so that it can apply force to collapse the implant (into the delivery configuration as shown in FIG. 1), or allow release of the implant into the expanded/deployed configuration shown in FIG. 4. The inserter may therefore include a portion that connects to the distal end of the implant by mating with the distal connector and a portion that connects with the proximal end of the implant by mating with the proximal connector.

The outer surface of the implant, and particularly the struts, may be smooth (and polished), or they may be rough, sharp, or tissue-penetrating. For example, the struts, or a portion of the struts, may include a tissue-cutting (and particularly cancellous bone cutting) region, or a tissue-gripping region. In some variations the implant is coated with a material, including a drug or other medicament, or a non-reactive material (e.g., a biocompatible material) or a reactive (e.g., cross-linkable) material.

In general, the struts may be made of any appropriate material. In some variations, the struts and other body regions are made of substantially the same material. Different portions of the stabilization device (including the struts) may be made of different materials. In some variations, the struts may be made of different materials (e.g., they may be formed of layers, and/or of adjacent regions of different materials, have different material properties). The struts may be formed of a biocompatible material or materials. It may be beneficial to form struts of a material having a sufficient spring constant so that the device may be elastically deformed from the deployed configuration into the delivery configuration, allowing the device to self-expand back to approximately the same deployed configuration. In some variation, the strut is formed of a shape memory material that may be reversibly and predictably converted between the deployed and delivery configurations. Thus, a list of exemplary materials may include (but is not limited to): biocompatible metals, biocompatible polymers, polymers, and other materials known in the orthopedic arts. Biocompatible metals may include cobalt chromium steel, surgical steel, titanium, titanium alloys (such as the nickel titanium alloy Nitinol), tantalum, tantalum alloys, aluminum, etc. Any appropriate shape memory material, including shape memory alloys such as Nitinol may also be used.

Other regions of the stabilization device may be made of the same material(s) as the struts, or they may be made of a different material. Any appropriate material (preferably a biocompatible material) may be used (including any of those materials previously mentioned), such as metals, plastics, ceramics, or combinations thereof. In variations where the devices have bearing surfaces (i.e. surfaces that contact another surface), the surfaces may be reinforced. For example, the surfaces may include a biocompatible metal. Ceramics may include pyrolytic carbon, and other suitable biocompatible materials known in the art. Portions of the device can also be formed from suitable polymers include polyesters, aromatic esters such as polyalkylene terephthalates, polyamides, polyalkenes, poly(vinyl) fluoride, PTFE, polyarylethyl ketone, and other materials. Various alternative embodiments of the devices and/or components could comprise a flexible polymer section (such as a biocompatible polymer) that is rigidly or semi rigidly fixed.

As mentioned, the implants (including the struts), may also include one or more coating or other surface treatment (embedding, etc.). Coatings may be protective coatings (e.g., of a biocompatible material such as a metal, plastic, ceramic, or the like), or they may be a bioactive coating (e.g., a drug, hormone, enzyme, or the like), or a combination thereof. For example, the stabilization devices may elute a bioactive substance to promote or inhibit bone growth, vascularization, etc. In one variation, the device includes an elutible reservoir of bone morphogenic protein (BMP).

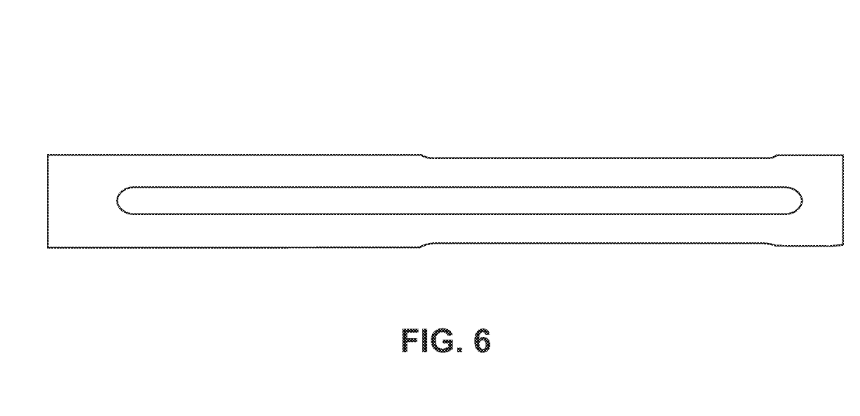

FIG. 4 illustrates a perspective view of the implant with bifurcated struts shown in FIGS. 1-3. This variation includes two arcing support struts 401, 401′ that are each divided along part of their length into two sub-struts 403, 405 and 403′ 405′. In FIG. 4 the struts are longitudinally symmetric, so that the support struts are approximately centered along the length of the body of the implant, and the sub-struts are approximately centered along the length of the body of the implant. In other variations, as illustrated in FIGS. 5-8, the struts are not centered, but are located towards the distal end of the implant (opposite the end of the implant that attached to the inserter).

The overall shape of the implant includes only a single expanded region (the middle region of the implant shown in FIG. 4). This may be advantageous because it allows support for the bone while maximizing the expandable height that can be achieved with the relatively small implant. This shape may also allow the implant to expand and contract in a controllable and predictable way.

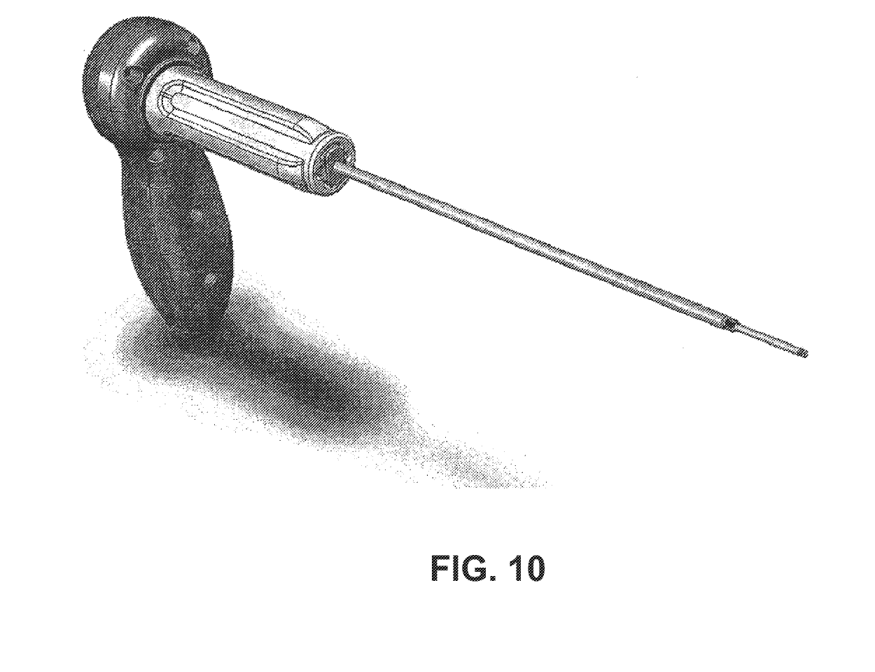

The variation of the implant shown in FIGS. 5-8 is similar to that shown in FIGS. 1-4, but the sub-struts are positioned toward one end (the distal end) of the implant. For example, in FIG. 5, the side perspective view of the implant illustrates a variation in which the major (support) struts and minor (sub) struts are formed by cutting into the tubular body. The implant is pre-biased into the expanded shape shown in FIG. 8. In this example, the sub-struts extend along a portion of the struts, but at the distal end of each strut. Thus only a portion of the support strut is bifurcated into sub-struts (e.g., less than 95%, 90%, 85%, 80%, 75%, 70%, 65%, 60%, 55%, 50%, 45%, 40%, 35%, 30%, etc.) of the support forms sub-struts.

The major/support strut may extend along only a portion of the elongate body of the implant. For example, the support strut may extend less than or approximately 95%, 90%, 85%, 80%, 75%, 70%, 65%, 60%, 55%, 50%, 45%, 40%, 35%, 30%, etc. of the length of the implant in the collapsed (delivery) configuration.

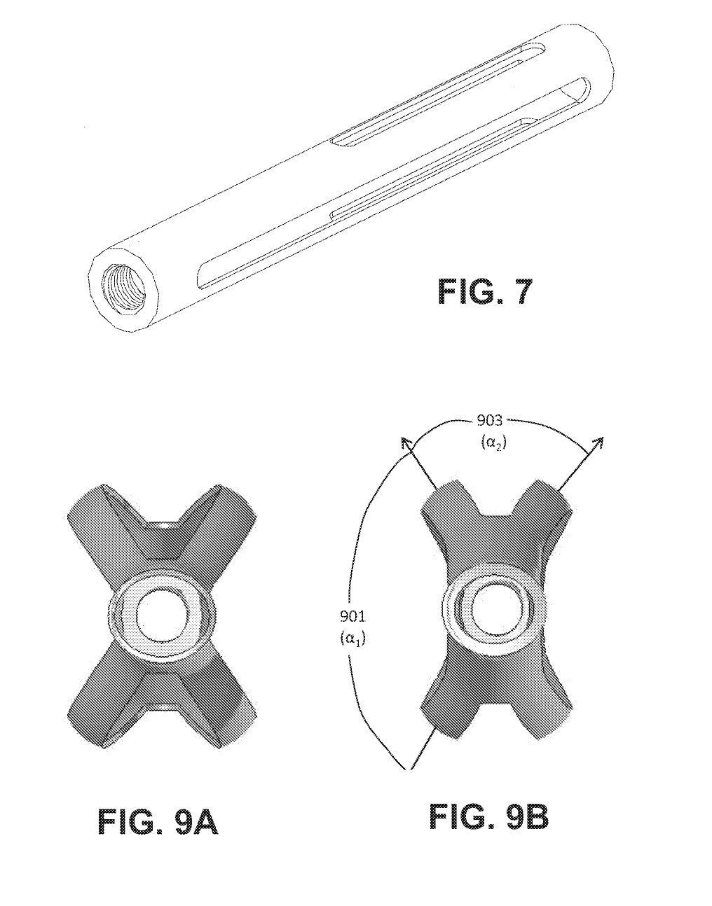

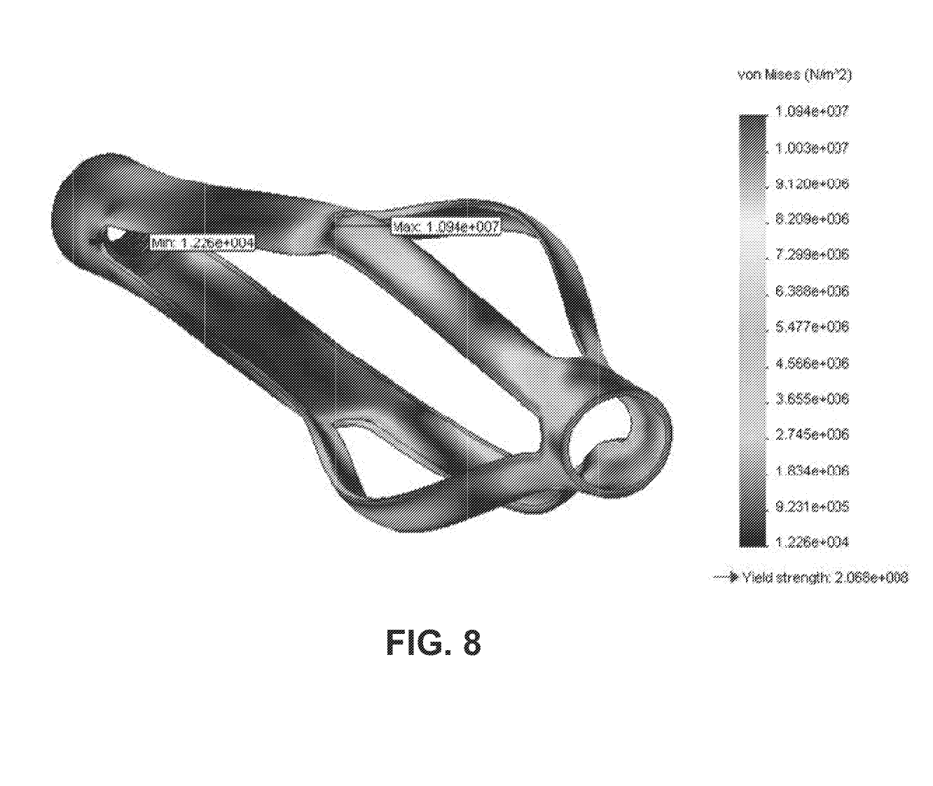

FIGS. 6 and 7 show side and perspective views of the implant shown in FIG. 6.

An expanded (e.g., deployed) configuration for the implant of FIGS. 5-7 is shown in FIG. 8. This figure is also marked by a pseudo-coloring indicating the stresses that the implant is capable of withstanding. The scale shown to the right of the figure correlates the pseudo-coloring in terms of the von Mises stress or equivalent tensile stress (a scalar stress value that can be computed from the stress tensor). A material is said to start yielding when its von Mises stress reaches a critical value known as the yield strength. The von Mises stress may be used to predict yielding of materials under any loading condition from results of simple uniaxial tensile tests. The von Mises stress satisfies the property that two stress states with equal distortion energy have equal von Mises stress.

FIG. 8 was calculated to determine the effect of the bifurcated shape in comparison to other implant shapes, including non-bifurcated shapes. Based this analysis, the bifurcated shape may have a greater reliability (e.g., lower failure rate) than other shapes, by allowing the distribution of stresses over the body of the implant in a way that prevents failure, particularly at the joints where the struts meet. As an implant that may be used within a patient for the lifetime of the patient, this may be very important. There may also be other advantages to the bifurcated struts compared to other implants having struts, including enhanced stability and width of expansion. The bifurcated struts may create a larger effective contact surface between internal ends of the vertebra, when the implant is used within cancellous bone of a vertebra.

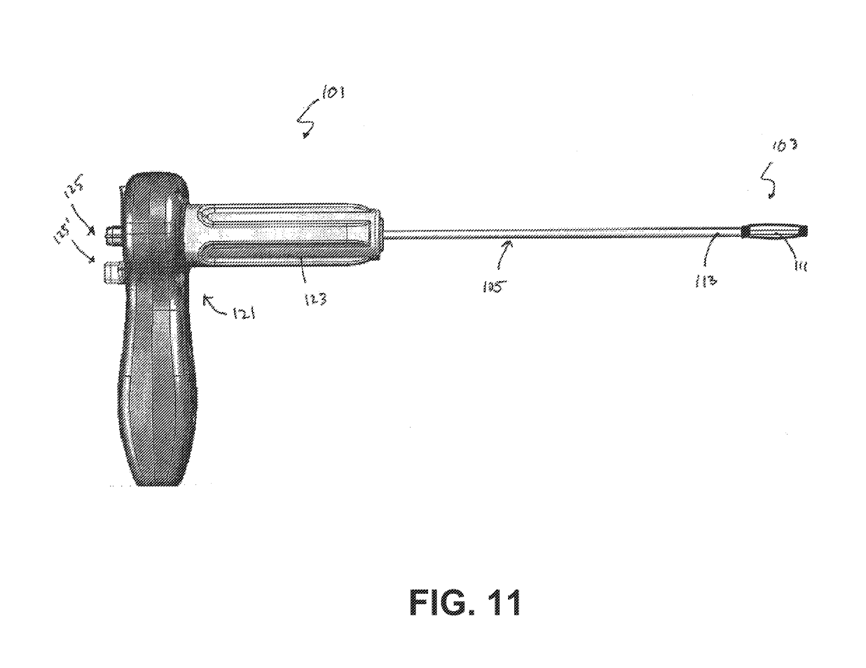

In general, the angle between adjacent struts and sub-struts in the bifurcated strut implants is different, as illustrated in FIGS. 9A and 9B. FIG. 9A shows an end view of the implant of FIG. 9 from the proximal end, and FIG. 9B shows the device viewed from the distal end. FIG. 9B has been labeled to show the major angle 901 (α₁, the angle between adjacent struts of different support struts) and the minor angle 903 (α₂, the angle between adjacent struts of the same support strut). The minor angle is typically less than 360°/(the number of sub-struts in the implant), while the major angle is typically greater than 360°/(the number of sub-struts in the implant).

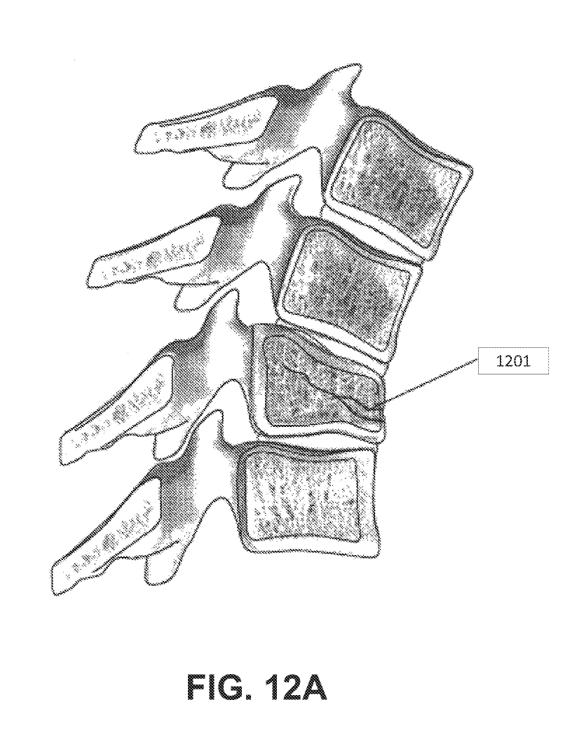

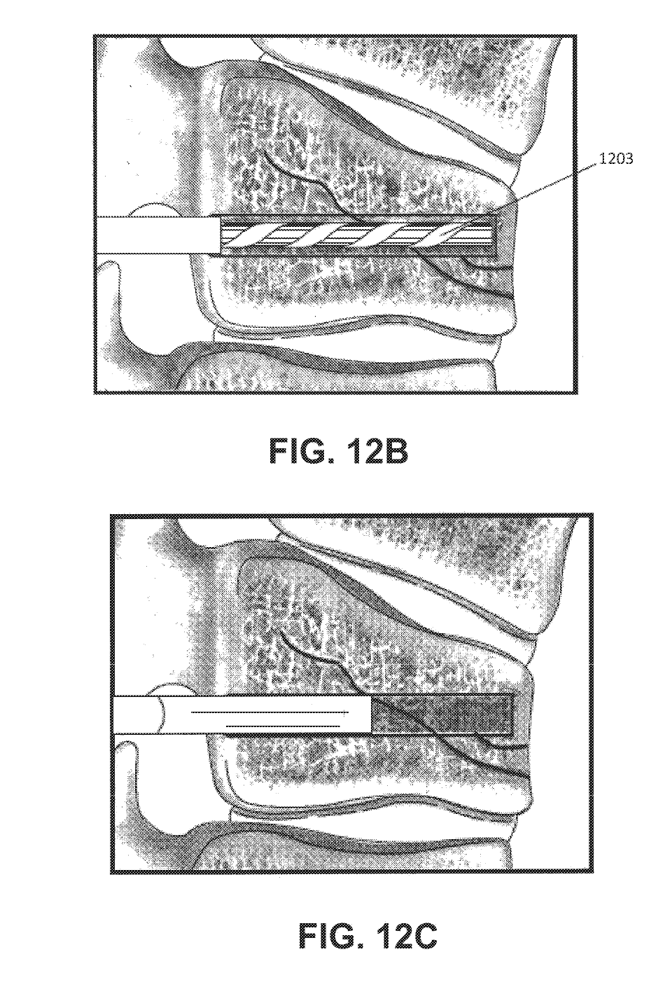

The device may be used with any appropriate inserter for inserting the device into the body, and particularly the bone. FIGS. 10 and 11 illustrate one variation of an inserter (which may also be referred to as an “implant applicator” or simply “applicator”) that may be used.

In some variations an applicator may include a handle and an elongate cannula region. An example of one variation of an applicator is shown in FIGS. 10 and 11. As labeled in FIG. 11, the applicator 101 includes a handle portion 107 and an elongate cannula 105, which may be referred to as a delivery device or as an elongate linkage member. An implant 103 is shown attached to the distal end of the applicator 101. In this example, the implant is held in a collapsed configuration by applying force from both ends of the implant. The elongate linkage member includes a second elongate member, inner member (rod) 111, and a first elongate member, outer member 113, that are movably (slideably) disposed relative to each other. In FIG. 11 the proximal end of the bone stabilization implant is releasably coupled to the outer member 113 (first elongate member) and the distal end of the implant is releasably coupled to the inner member 111 (second elongate member). The applicator 101 may separately control the relative motion of the proximal and distal end of the implant (which is pre-biased to self-expand to a delivery configuration) by controlling the relative motions of the outer cannula 113 and the inner member 111 at the handle 121. In this example, the handle includes a ratchet mechanism 123 (e.g., a rotary gear including a pawl, not visible in FIG. 1) and a number of controls 125,125′ for directing the motion of the applicator.

As mentioned in some variations, the inserter includes a lock or locks that hold the stabilization device in a desired configuration. For example, the inserter may be locked so that the stabilization device is held in the delivery configuration (e.g., by applying force between the distal and proximal ends of the stabilization device). In an inserter such as the one shown in FIGS. 10 and 11, for example, a lock may secure the first elongate member to the second elongate member so that they may not move axially relative to each other.

FIG. 10 shows an inserter without an attached stabilization implant, and FIG. 11 shows the same inserter with an implant attached. The first and the second elongate members of the inserter are configured coaxially (as a rod and shaft) that may be moved axially and rotationally independently of each other. The distal end of the stabilization device includes a releasable attachment that is configured as a threaded region which mates with a threaded complementary attachment at the distal end of the structure.

The proximal ends of the coaxial first and second elongated members may also include grips. These grips (not shown) may be grasped directly by a person (e.g., a physician, technician, etc.) using the device, or they may be connected to a handle. Thus, in some variations one or both grips are ‘keyed’ to fit into a handle, so that they can be manipulated by the handle. A grip may be a knob attached to the first and/or second elongated member. This knob may also be used to move the elongate members of the inserter (e.g., to rotate them), or to otherwise hold it in a desired position. The knob may be shaped and/or sized (e.g., to fit a handle). In some variations this knob 741 is a keyed member that is secured to the handle. This keyed member may be configured to secure within a handle so and may help orient the device (including the implant) and the handle, and may sever to secure the cannula in the handle. The keyed member may have an outer shape (e.g., rectangular, etc.) that locks the relative motion of all or a portion of the handle with respect to the outer member.

Any of the inserters described herein may include, or may be used with, a handle. A handle may allow a user to control and manipulate an inserter. For example, a handle may conform to a subject's hand, and may include other controls, such as triggers or the like. Thus, a handle may be used to control the relative motion of the first and second elongate members of the inserter, or to release the connection between the stabilization device and the inserter, or any of the other features of the inserter described herein.

In general, any of the implants described herein may be used as part of a system or kit including any appropriate applicator for controlling the proximal and distal ends of the implant and allowing it to be inserted into the body. A system may also include additional components, including instructions for use or operation (in any appropriate format, including written, visual and electronic), multiple implants, multiple implant inserters, or the like. For example, an implant may be packaged sterile with an implant inserter, or at least the first and second slideably coupled elongate members of the implant inserter pre-attached to an implant. Different sized implants may be used, and a system or kit may include multiple sizes. One or more handle portions may be used. The handle may be reusable or disposable.

In operation, the implants described herein may be inserted into a patient's body in the collapsed configuration (by applying stress to maintain the device in the collapsed configuration) and allowed to expand (e.g. by self-expanding in a controlled or uncontrolled manner) into the deployed and expanded configuration. In some variations the device may be radially oriented (e.g., using visual means such as floro, direct visualization, or the like) so that the bifurcated support struts face the “top” and “bottom” of the vertebra, relative to the patient's body. This may allow the bifurcated struts in a single support strut to spread apart and provide support.

As mentioned above, any of the devices described herein may be used to repair a bone. A method of treating a bone using the devices describe herein typically involves delivering an implant having bifurcated struts (e.g., the a self-expanding stabilization devices described herein) within a cancellous bone region, and allowing the device to expand within the cancellous bone region so that a cutting surface of the implant cuts through the cancellous bone and the bifurcated struts come to support (and possibly distract) the cortical bone.

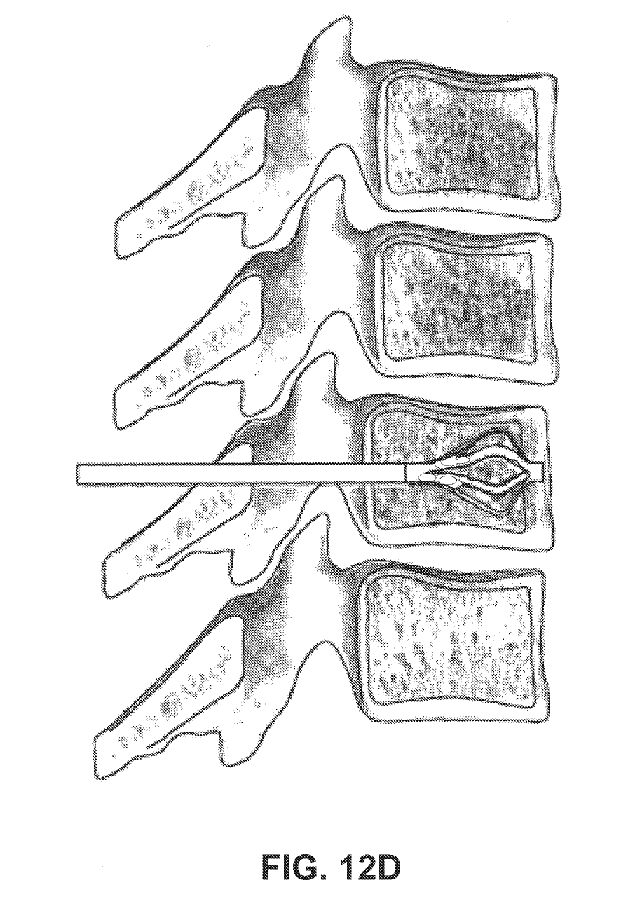

For example, as illustrated in FIGS. 12A-12D, the implants described herein may be used to repair a compression fracture 1201 in spinal bone. The spinal vertebras are normally aligned, distributing pressure across each vertebra. In a compression fracture the bone may be fractured, and should be restored to its uninjured position by expanding (also referred to as distracting) the vertebra so that the shape of the cortical bone is restored. This may be achieved by inserting and expanding one of the stabilization devices described herein. In order to insert the stabilization device, the damaged region of bone must be accessed.

As mentioned above, an introducer (or access cannula) and a trocar may be used to insert the access cannula adjacent to the damaged bone region. Any of the steps described herein may be aided by the use of an appropriate visualization technique. For example, a fluoroscope may be used to help visualize the damaged bone region, and to track the p of inserting the access cannula, trocar, and other tools. Once the access cannula is near the damaged bone region, a bone drill 1203 may be used to drill into the bone, as shown in FIG. 12B.

The drill may enter the cancellous bony region within the vertebra. After drilling into the vertebra to provide access, the drill is removed from the bone and the access cannula is used to provide access to the damaged vertebra, by leaving the access cannula in place, providing a space into which the stabilization device may be inserted in the bone. An implant (“stabilization device”), attached to an inserter and held in the delivery configuration, may then be inserted into the damaged vertebra, as shown in FIG. 12C.

Once in position within the vertebra, the stabilization device is allowed to expand (by self-expansion) within the cancellous bone of the vertebra. In some variations, the device may fully expand, cutting through the cancellous bone and pushing against the cortical bone with a sufficient restoring force to correct the compression. However, in some variations, the force generated by the device during self-expansion is not sufficient to distract the bone, and the inserter handle may be used (e.g., by applying force to the handle, or by directly applying force to the proximal end of the inserter) to expand the stabilization device until the cortical bone is sufficiently distracted.

Once the stabilization device has been positioned and is expanded, it may be released from the inserter. In some variations, it may be desirable to move or redeploy the stabilization device, or to replace it with a larger or smaller device. If the device has been separated from the inserter (e.g., by detaching the removable attachments on the stabilization device from the cooperating attachments on the inserter), then it may be reattached to the inserter. Thus, the distal end of the inserter can be coupled to the stabilization device after implantation. The inserter can then be used to collapse the stabilization device back down to the delivery configuration, and the device can be withdrawn or re-positioned.

As mentioned above, a cement or additional supporting material may also be used to help secure the stabilization device in position and to repair the bone. For example, bone cement may be used to cement a stabilization device in position. Although in some variations the addition of the stabilization device may be sufficient to repair the bone, it may also be desirable to add a cement, or filler to help secure the repair. This may also help secure the device in position, and may help close the surgical site.

For example, a fluent bone cement may be added to the cancellous bone region around implant. This cement will flow through the channels of trebeculated (cancellous) bone, and secure the implant in position. This is shown in greater detail in the enlarged region. This bone cement or filler can be applied using the delivery cannula (e.g., through a cement cannula), and allowed to set.

The devices and methods for treating vertebral bodies describes above may be used for the implantation of an implant through a pedicle into the cancellous bone interior of a vertebral body. The implant may be longitudinally shortened during implantation, as mentioned. The expansion of the implant may be controlled so that only the proximal end is moved relative to the bone, while the implant is inserted.

Further, the implant may be sized and configured such that self-expansion takes the device to an appropriate dimension for the vertebral body. Thus, as the device approaches its final expanded dimension, it presses the surface outwardly so as to restore the height and volume of the vertebral body toward the dimensions of the vertebral body prior to the fracture.

Methods of using the bifurcated implants and applicators (and systems including them) may include a step of selecting devices appropriate in form, shape, and size for each implantation site. Thus, in some variations the applicator or inserter devices described herein may be configured so that they may be used with implants of different sizes (both length and/or widths). For example, the devices may be configured so that the relative movement and separation of the inner and outer members spans a variety of sizes (e.g., lengths) of the bone stabilization implants from expanded to collapsed lengths. In some variations the handles include a limiter that prevents overexpansion of the applicator when coupled to an implant.

While preferred embodiments of the present invention have been shown and described herein, such embodiments are provided by way of example only. Numerous variations, changes, and substitutions are possible without departing from the invention. Thus, alternatives to the embodiments of the invention described herein may be employed in practicing the invention. The exemplary claims that follow help further define the scope of the systems, devices and methods (and equivalents thereof).

What is claimed is:

1. A bone stabilization implant pre-biased to self-expand from a collapsed delivery configuration to an expanded deployed configuration within cancellous bone, the bone stabilization implant comprising:
an elongated body having a proximal end and a distal end and an inner passage extending through the elongated body from the proximal end to a region of the distal end;
a plurality of bifurcated support struts configured to extend from the elongated body, wherein each bifurcated support strut is configured to extend from the elongated body in the deployed configuration and to separate into two or more sub-struts extending longitudinally from a portion of the bifurcated support strut;
a proximal attachment region having a first releasable attachment configured to attach to an inserter; and
a distal attachment region having a second releasable attachment configured to attach to the inserter.

2. The bone stabilization implant of claim 1, wherein the proximal attachment region is within the inner passage of the elongate body.

3. The bone stabilization implant of claim 1, wherein the proximal attachment region is a threaded region within the inner passage of the elongate body.

4. The bone stabilization implant of claim 1, wherein the distal attachment region is within the inner passage of the elongate body.

5. The bone stabilization implant of claim 1, wherein the distal attachment region is a threaded region within the inner passage of the elongate body.

6. The bone stabilization implant of claim 1, wherein the proximal and distal attachment regions comprise threaded regions within the inner passage of the elongate body that are threaded in opposite directions.

7. The bone stabilization implant of claim 1, wherein the sub-struts are configured to extend from the elongate body asymmetrically relative to the length of the elongate body.

8. The bone stabilization implant of claim 1, wherein the sub-struts are arranged to extend more from the distal end of the length of the elongate body.

9. The bone stabilization implant of claim 1, wherein the sub-struts extend longitudinally from less than 90% of the length of the strut.

10. A bone stabilization implant pre-biased to self-expand from a collapsed delivery configuration to an expanded deployed configuration within cancellous bone, the bone stabilization implant comprising:
an elongate body having a proximal end and a distal end and an inner passage extending through the elongate body from the proximal end to a distal end region;
a plurality of bifurcated support struts configured to extend from the elongate body, wherein each bifurcated strut is configured to extend from the elongate body in the deployed configuration and separate into two or more sub-struts extending longitudinally from less than 90% of the length of the strut;
a proximal attachment region in the inner passage of the elongate body having a first releasable attachment configured to attach to an inserter; and
a distal region having a second releasable attachment configured to attach to the inserter.

11. A bone stabilization implant pre-biased to expand from a collapsed delivery configuration to an expanded deployed configuration within cancellous bone, the bone stabilization implant comprising:
an elongate body having a proximal end and a distal end and an inner passage extending through the elongate body from the proximal end to a distal end region;
a plurality of bifurcated support struts configured to extend from the elongate body and separate along part of their length into two or more sub-struts, wherein the angles between adjacent sub-struts on each bifurcated support strut is less than the angle between adjacent sub-struts on different bifurcated support struts;
a proximal attachment region having a first releasable attachment configured to attach to an inserter; and
a distal attachment region having a second releasable attachment configured to attach to the inserter.

12. The bone stabilization implant of claim 11, wherein the proximal and distal attachment regions comprise threaded regions within the inner passage of the elongate body that are threaded in opposite directions.

13. The bone stabilization implant of claim 11, wherein the sub-struts are configured to extend from the elongate body asymmetrically relative to the length of the elongate body.

14. The bone stabilization implant of claim 11, wherein the sub-struts are arranged to extend more from the distal end of the length of the elongate body.

15. The bone stabilization implant of claim 11, wherein the sub-struts extend longitudinally from less than 90% of the length of the strut.

16. A stabilization system comprising:
an implant inserter having a first elongate member and a second elongate member, wherein the first and second elongate member are slideably coupled; and
a stabilization implant pre-biased to expand from a collapsed delivery configuration to an expanded deployed configuration within cancellous bone, the bone stabilization implant comprising:
an elongate body having a proximal end and a distal end and an inner passage extending through the elongate body from the proximal end to a distal end region;
a plurality of bifurcated support struts configured to extend from the elongate body and separate along part of their length into two or more sub-struts;
a proximal attachment region configured to releasable attach to the first elongate member of the inserter; and
a distal attachment region configured to releasably attach to the second elongate member of the inserter.

17. The system of claim 16, wherein inserter comprises a lock configured to axially lock the first elongate member relative to the second elongate member.

18. The system of claim 16, wherein the first elongate member of the inserter is slideably disposed within the second elongate member.

19. A method of treating a bone with a pre-biased stabilization implant having an elongate body with a proximal and distal end and a plurality of bifurcated support struts configured to extend from the elongate body and separate into two or more sub-struts, the method comprising:
delivering the pre-biased stabilization implant within a cancellous bone in a collapsed configuration; and
allowing the implant to self-expand within the cancellous bone so that the sub-struts cuts through the cancellous bone and secure the implant within the bone.

20. The method of claim 19, wherein the step of allowing the device to self-expand comprises allowing the proximal end of the implant to move towards the distal end of the implant while the distal end of the implant remains relatively fixed with respect to the bone.
